(19) United States
(12) Patent Application Publication
(10) Pub. No.: US 2025/0009514 A1
(43) Pub. Date: Jan. 9, 2025

(54) DISPOSABLE FURLOW INSERTION DEVICE WITH COLLAPSIBLE PUSHER

(71) Applicant: Boston Scientific Medical Device Limited, Galway (IE)

(72) Inventors: Anurag Sharma, Tundla (IN); Vivek Sharma, Kanpur (IN)

(21) Appl. No.: 18/760,670

(22) Filed: Jul. 1, 2024

Related U.S. Application Data
(60) Provisional application No. 63/512,146, filed on Jul. 6, 2023.

(51) Int. Cl. A61F 2/26

(52) U.S. Cl. CPC A61F 2/26

(57) ABSTRACT

According to a general aspect, an insertion device includes an elongate member defining a lumen extending from a first end of the elongate member to a second end of the elongate member, and a pusher having a first end portion. The first end portion of the pusher is configured to be disposed within the lumen defined by the elongate member and move from a first position within the lumen defined by the elongate member to a second position within the lumen defined by the elongate member. The first end portion of the pusher defines a slot.

### CROSS-REFERENCE TO RELATED APPLICATION

This application claims priority to U.S. Provisional Patent Application No. 63/512,146, filed on Jul. 6, 2023, entitled “DISPOSABLE FURLOW INSERTION DEVICE WITH COLLAPSIBLE PUSHER”, the disclosure of which is incorporated by reference herein in its entirety.

### TECHNICAL FIELD

This disclosure relates generally to devices for insertion of bodily implants and, more specifically, to disposable insertion devices for bodily implants, such as penile prostheses.

### BACKGROUND

One treatment for chronic, organic, male erectile dysfunction is the implantation of a penile prosthesis that mechanically erects the penis. Such prostheses can have different physical constructions (e.g., materials, mechanical structure and function, etc.) and configurations (e.g., physical dimensions, etc.). For instance, such a penile prosthesis can be in the form of a cylinder (e.g., malleable, inflatable, etc.) that is selected based on the physical requirements of an intended recipient (a patient). For instance, an appropriate penile prosthesis can be implanted within a corpus cavernosum (corpus cavernosa) of a patient's penis by medical personnel that are trained and knowledgeable regarding the implantation and use of penile prostheses.

Insertion devices (e.g., Furlow insertion devices) can be used to facilitate the insertion of a penile prosthesis into the corpus cavernosum of a patient. Such Furlow insertion devices can be used for measurement of an insertion length within the corpus cavernosum, as well as for facilitating insertion of a penile prosthesis by deploying a suture-carrying needle, where the needle and/or suture are used to introduce (draw, pull, insert, implant, etc.) the prosthesis into the corpus cavernosum.

### SUMMARY

According to a general aspect, an insertion device includes an elongate member defining a lumen extending from a first end of the elongate member to a second end of the elongate member, and a pusher having a first end portion. The first end portion of the pusher is configured to be disposed within the lumen defined by the elongate member and move from a first position within the lumen defined by the elongate member to a second position within the lumen defined by the elongate member. The first end portion of the pusher defines a slot.

In some implementations, the first end portion of the pusher defines a receiving portion. In some implementations, the first end portion of the pusher defines a receiving portion configured to receive a portion of a needle. In some implementations, the pusher has a second end portion opposite the first end portion, the second end portion includes a handle portion. In some implementations, the pusher has a second end portion opposite the first end portion, the second end portion includes a handle portion, the handle portion being configured to extend from the lumen defined by the elongate member. In some implementations, the pusher has a second end portion opposite the first end portion, the second end portion includes a handle portion, the handle portion being configured to extend from the lumen defined by the elongate member while the first end portion of the pusher is disposed within the lumen defined by the elongate member.

In some implementations, the slot defined by the first end portion of the pusher extends in a direction parallel to a longitudinal axis of the pusher. In some implementations, the slot defined by the first end portion of the pusher is a first slot, the first end portion of the pusher defining a second slot. In some implementations, the slot defined by the first end portion of the pusher is a first slot, the first end portion of the pusher defining a second slot, the first slot extends in a direction parallel to a longitudinal axis of the pusher, the second slot extends is a direction parallel to the longitudinal axis of the pusher.

In some implementations, the first end portion of the pusher defines a plurality of slots. In some implementations, the slot defined by the first end portion of the pusher is a first slot, the first end portion of the pusher defining a second slot, a third slot, and a fourth slot.

In some implementations, the first end portion of the pusher is configured to be placed in an expanded configuration and a collapsed configuration. In some implementations, the first end portion of the pusher is configured to be placed in an expanded configuration and a collapsed configuration, the first end portion of the pusher being configured to be placed in the collapsed configuration in response to the first end portion of the pusher contacting an inner diameter of the elongate member.

In some implementations, the first end portion of the pusher includes a first section disposed on a first side of the slot and a second section disposed on a second side of the slot, the first end portion of the pusher is configured to be placed in an expanded configuration and a collapsed configuration, the first section of the pusher being disposed closer to the second section of the pusher when the first end portion of the pusher is disposed in the collapsed configuration than when the first end portion of the pusher is disposed in the expanded configuration.

In some implementations, the elongate member defines a slot.

According to another aspect, a kit includes a bodily implant; a delivery needle configured to be coupled to the bodily implant; and a delivery device having an elongate member defining a lumen extending from a first end of the elongate member to a second end of the elongate member, and a pusher having a first end portion, the first end portion of the pusher being configured to be disposed within the lumen defined by the elongate member and move from a first position within the lumen defined by the elongate member to a second position within the lumen defined by the elongate member, the first end portion of the pusher defining a slot.

In some implementations, the elongate member defines a slot configured to receive at least a portion of the delivery needle. In some implementations, the slot defined by the first end portion of the pusher extends in a direction parallel to a longitudinal axis of the pusher. In some implementations, the slot defined by the first end portion of the pusher is a first slot, the first end portion of the pusher defining a second slot. In some implementations, the slot defined by the first end portion of the pusher is a first slot, the first end portion of the pusher defining a second slot, the first slot extends in a direction parallel to a longitudinal axis of the pusher, the second slot extends is a direction parallel to the longitudinal axis of the pusher. In some implementations, the first end portion of the pusher defines a plurality of slots.

### BRIEF DESCRIPTION OF THE DRAWINGS

FIG. 1 schematically illustrates a system according to an implementation.

FIG. 2 is a perspective view of an insertion device according to an implementation.

FIGS. 3 and 4 are cross-sectional views of the insertion device of FIG. 2.

FIGS. 5 and 6 are cross sectional views of a portion of the insertion device of FIG. 2.

FIG. 7 is a perspective view of an elongate member of the insertion device of FIG. 2.

FIG. 8 is a cross-sectional view of the elongate member of FIG. 7.

FIG. 9 is a side view of a coupler of the insertion device of FIG. 2.

FIG. 10 is a cross-sectional view of the coupler of FIG. 9.

FIG. 11 is a perspective view of a pusher of the insertion device of FIG. 2.

FIG. 12 is a perspective view of an end portion of the pusher or FIG. 11.

FIG. 13 is a side view of the insertion device of FIG. 2.

FIG. 14 is a cross-sectional view of the insertion device taken along line A-A of FIG. 13.

FIG. 15 is a cross-sectional view of the insertion device taken along line B-B of FIG. 13.

FIG. 16-18 illustrate a method of using an insertion device according to an implementation.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as insertion devices for penile prostheses or other bodily implants. The term patient may hereafter be used for a person who benefits from the medical device or the methods disclosed in the present disclosure. For example, the patient can be a person whose body is implanted using the medical device or benefits from the methods disclosed for operating the medical devices of the present disclosure. For example, in some embodiments, the patient may be a human male, a human female, or any other mammal.

The terms proximal and distal described in relation to various devices, apparatuses, and components as discussed in the subsequent text of the present disclosure are referred to using a point of reference. The point of reference, as used in this description, is a perspective of a person who uses the disclosed insertion devices to implant a bodily implant, such as a penile prosthesis. The person may be a surgeon, a physician, a nurse, a doctor, a technician, and the like who may perform the implantation procedure. The term proximal refers to an area or portion that is closer or closest to the person during the implantation procedure. The term distal refers to an area or portion that is farther from, or farthest from the person.

The embodiments discussed herein may provide improvements to penile prosthesis insertion devices (e.g., Furlow insertion devices, Furlow insertion tools, insertion tools, devices, tools, etc.). For example, such insertion devices can be configured to be used in a single procedure and then discarded or disposed of. Some such insertion devices may have components that are coupled together and that when removed from each other would destroy the insertion device.

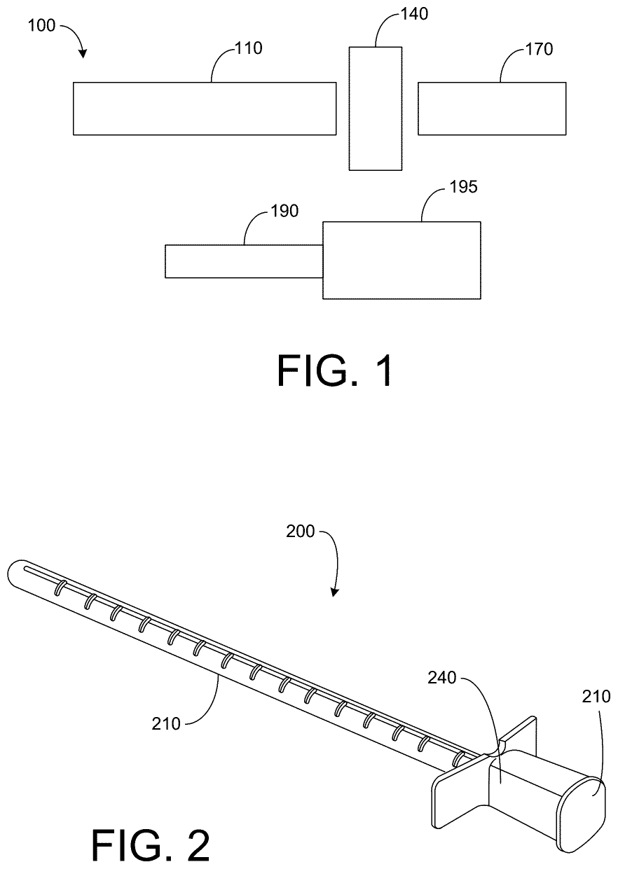

FIG. 1 schematically illustrates a system according to an implementation. The system includes a delivery or insertion device 100, an implant 195, and a needle 190 coupled to the implant 195. In some implementations, the implant 195 may be a penile implant or another type of implant that is configured to be placed in the pelvic region of the patient. In other implementations, the implant 195 is configured to be placed within the body of the patient at a different location. In some implementations, the needle 190 is coupled to the implant 195 via a suture. In other implementations, the needle 190 is directly coupled to the implant 195 or is coupled to the implant 195 via another mechanism.

The insertion device 100 includes an elongate member 110, a coupler 140, and a pusher or an obturator 170. The elongate member 110 is coupled to the coupler 140 and the pusher or obturator 170 is coupled to the coupler 140. In some implementations, the elongate member 110 is fixedly coupled to the coupler 140. In some implementations, the obturator 170 is moveably, such as slidably, coupled to the coupler 140. The pusher or obturator 170 has a portion that is configured to be disposed within a lumen defined by the elongate member 110.

In use, the needle 190 or a portion of the needle 190 may be placed or disposed within the lumen defined by the elongate member. The delivery device 100 including the needle 190 may be placed within the body of the patient to a desired location. The pusher or obturator 170 may then be advanced or moved distally within the lumen defined by the elongate member 110. The pusher or obturator 170 is configured to engage the needle 190 and advance the needle 190 out of the lumen defined by the elongate member 110. The delivery device 100 may then be removed from the body of the patient and the needle can then be pulled or otherwise moved within the body of the patient until the implant 195 is disposed at a desired location.

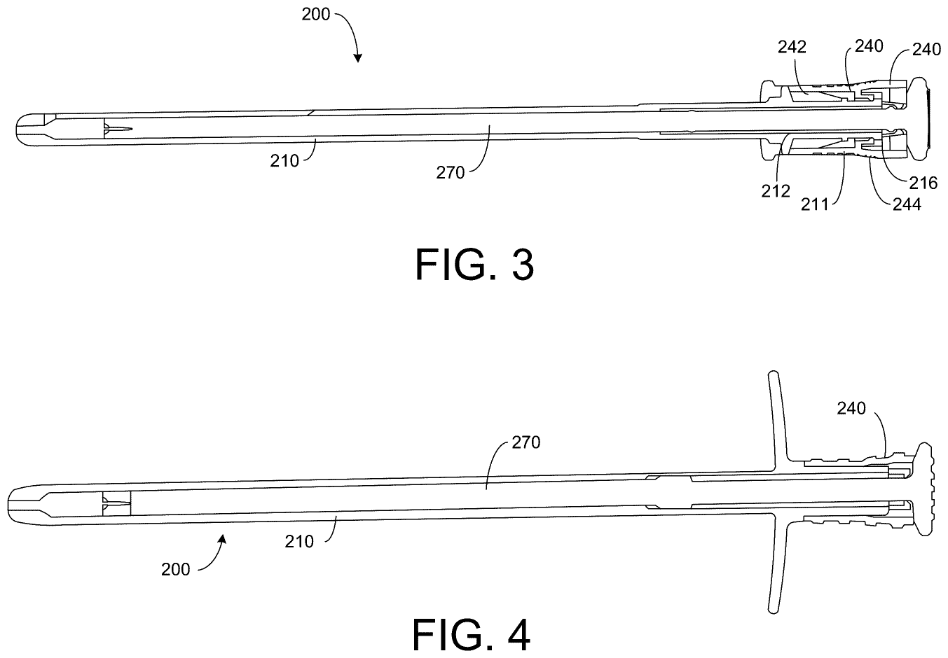

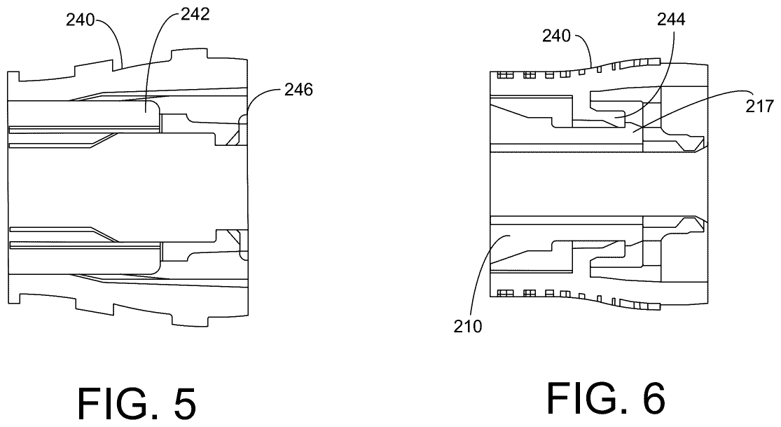

FIGS. 2-6 illustrate an insertion device 200 according to an implementation. FIG. 2 is a perspective view of the insertion device 200. FIGS. 3 and 4 are cross-sectional views of the insertion device. FIGS. 5 and 6 are cross sectional views of a portion of the insertion device.

The insertion device 200 includes an elongate member 210, a coupler 240, and a pusher or obturator 270. The elongate member 210 is coupled to the coupler 240 and the pusher or obturator 270 is coupled to the coupler 240. In the illustrated implementation, the elongate member 210 is fixedly coupled to the coupler 240.

As best illustrated in FIG. 3-6, the coupler 240 defines a lumen 242. A proximal end portion 212 of the elongate member 210 is configured to be disposed within the lumen 242 defined by the coupler 240. The proximal end portion 212 of the elongate member 210 includes an engagement portion or engagement member 217. The engagement portion or member 217 extends from an outer surface 211 of the elongate member 210. The coupler 240 also includes an engagement member 244. The engagement member 244 of the coupler 240 extends into the lumen 242 defined by the coupler 240. The engagement member 244 of the coupler 240 is configured to engage or interact with the elongate member 210 to couple the elongate member 210 to the coupler 240. As best illustrated in FIG. 6, the engagement member 244 of the coupler 240 is configured to engage or interact with the engagement member 217 of the elongate member 210. In some implementations, the engagement member 244 of the coupler 240 is configured to bend or flex to provide a snap type coupling to the elongate member 210. In other implementations, the elongate member 210 is coupled to the coupler via a different mechanism or different type of coupling.

As best illustrated in FIGS. 3-5, in the illustrated implementation, the pusher or obturator 270 is movably coupled to the coupler 240. At least a portion of the pusher or obturator 270 is disposed within the lumen 242 defined by the coupler 240 and is configured to slide, translate, or otherwise move with respect to the coupler 240. In the illustrated implementation, the coupler 240 includes an engagement member 246. As will be discussed in more detail below, the engagement member 246 extends into the lumen 242 defined by the coupler 240 and is configured to engage or contact a portion of the pusher or obturator 270.

At least a portion of the pusher or obturator 270 extends into a lumen 216 defined by the elongate member 210. The pusher or obturator 270 is configured to slide or otherwise move within the lumen 216 defined by the elongate member 210.

In use, a needle that is coupled to a bodily implant may be placed or disposed within the lumen 216 defined by the elongate member 210. The delivery device 200 including the needle may be placed within the body of the patient to a desired location. The pusher or obturator 270 may then be advanced or moved distally within the lumen 216 defined by the elongate member 210. The pusher or obturator 270 is configured to engage the needle and advance the needle out of the lumen 216 defined by the elongate member 210. The delivery device 200 may then be removed from the body of the patient and the needle can then be pulled or otherwise moved within the body of the patient until the implant is disposed or located at a desired location.

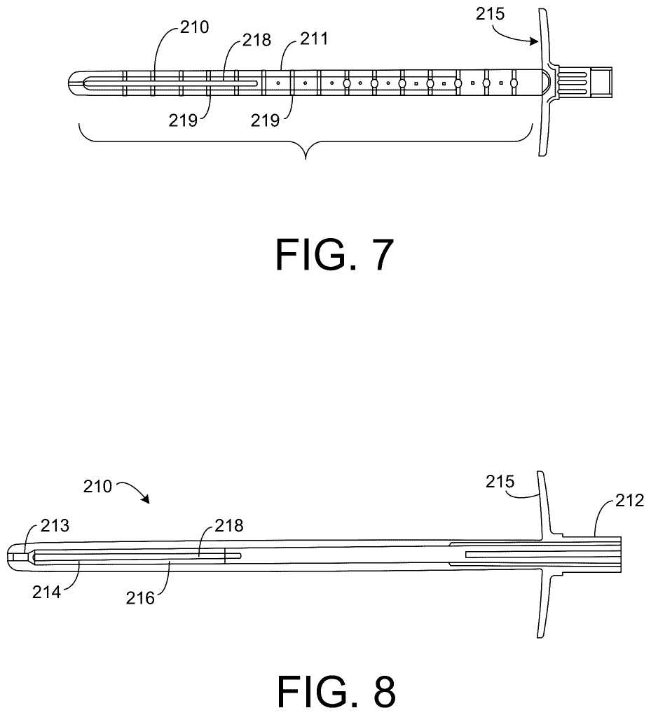

FIGS. 7 and 8 illustrate the elongate member 210. The elongate member 210 includes or defines a slot 218. The slot 218 is disposed at the distal end portion 214 of the elongate member 210 and communicates with the lumen 216 defined by the elongate member 210. The slot 218 is configured to receive the needle to facilitate the placement of the needle into the lumen 216 defined by the elongate member 210. The elongate member 210 also includes a tapering or tapered portion 213 of the lumen 216 at the distal end portion 214 of the elongate member 210. In some implementations, the tapering or narrowing of the lumen 216 (the inner diameter of the lumen gets smaller towards the end of the elongate member 210) helps eject or dispose of the needle more accurately as desired by the user. For example, the narrowing or tapering of the lumen 216 of the elongate member 210 may limit the or minimize the exit angle of the needle from the lumen 216.

In the illustrated implementation, the elongate member 210 includes a handle portion 215. The handle portion 215 extends from an outer surface 211 of the elongate member 210. The handle portion 215 may be grasped by the user during the insertion of the delivery device 200 into the body of the patient and during the advancement or movement of the pusher or obturator 270 with respect to the elongate member 210.

The elongate member 210 includes measurement markings 219. The measurement markings 219 may be configured to indicate to the user how far the elongate member 210 (or the delivery device 200) has been inserted into the body of the patient. In some implementations, the measurement markings 219 are laser etched onto the material of the elongate member 210. In such implementations, the surface of the elongate member 210 that includes the measurement markings 219 may be smooth. In other implementations, the measurement markings 219 may be formed in a different manner. For example, the measurement markings 219 may be painted, colored, or otherwise marked on the surface of the elongate member 210.

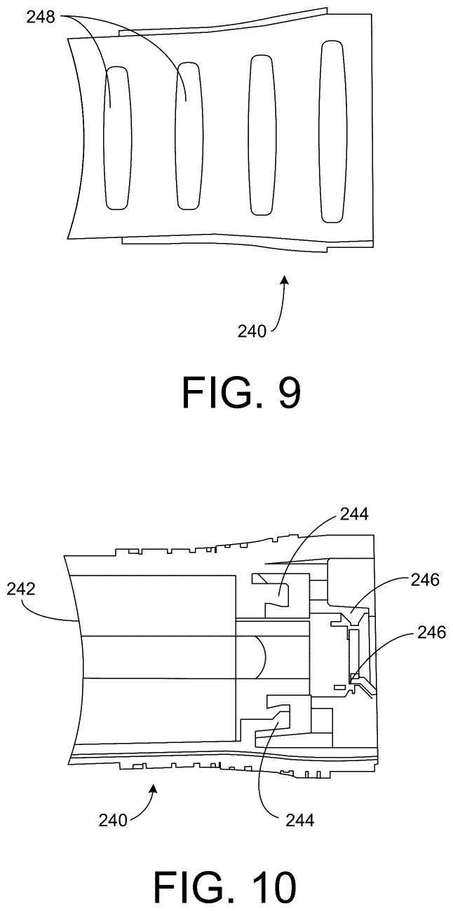

FIGS. 9 and 10 illustrate the coupler 240. In the illustrated implantation, the coupler 240 defines a lumen 242. The lumen 242 is configured to receive at least a portion of the elongate member 210 and at least a portion of the obturator 270. The coupler includes an engagement member 244 that is configured to engage the elongate member 210 to couple the elongate member 210 to the coupler 240. The engagement member 244 extends into the lumen 242 defined by the coupler 240. In some implementations, the engagement member 244 is configured to bend or flex.

The coupler includes an engagement member 246. The engagement member 246 extends into the lumen 242 defined by the coupler 240 and is configured to engage the pusher or obturator 270. In some implementations, the engagement member 246 is configured to contact or engage the pusher or obturator 270 to help prevent the pusher or obturator 270 from being removed from the coupler 240. Accordingly, the pusher or obturator 270 may move or slide within the lumen 242 defined by the coupler 240 but in some implementations, may not be removed entirely from the lumen 242 of the coupler 240.

In the illustrated implementation, the coupler 240 includes ridges or projections 248 that extend from an outer surface of the coupler 240. The ridges or projections 248 may facilitate the grasping or gripping of the coupler 240 or the delivery device 200 during use of the device.

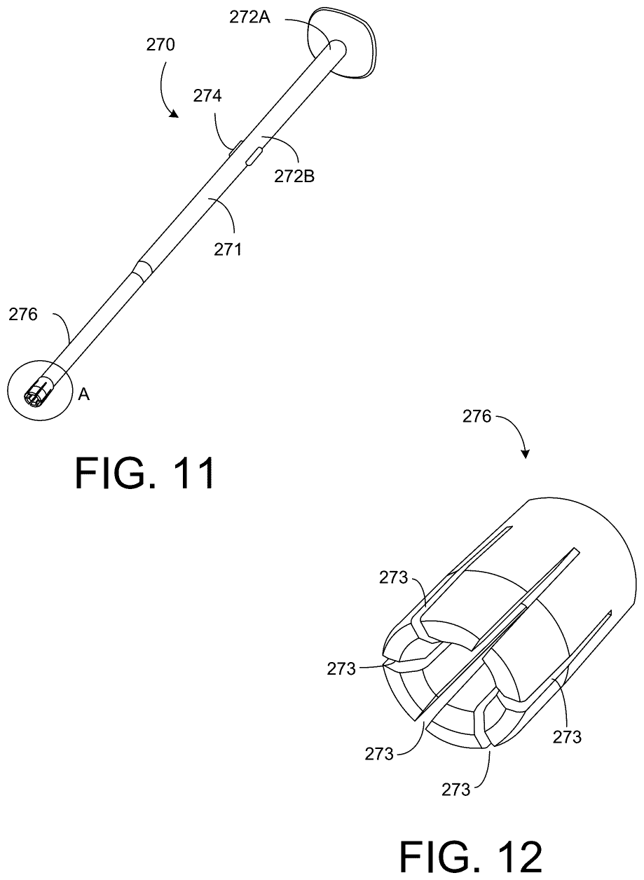

FIGS. 11 and 12 illustrate the pusher or obturator 270 of the delivery device 200. The pusher or obturator 270 includes a distal end portion 276 and a handle portion disposed opposite the distal end portion 276. The pusher or obturator 270 includes ramps or bumps 272A and 272B. The ramps or bumps extend from an outer surface 271 of the obturator 270 and are configured to engage the coupler 240. For example, in some implementations, the ramps or bumps 272A and 272B are configured to engage the engagement member 246 of the coupler 240. For example, in the illustrated implementation, the proximal ramps 272A may engage the engagement member 246 of the coupler 240 when the pusher or obturator 270 is placed in its distal most position. The distal ramp 272B may be configured to engage the engagement member 246 of the coupler 240 when the obturator is placed in its proximal most position. Additionally, in some implementations, the distal ramps 272B may engage the engagement member 246 of the coupler 240 to help prevent the removal of the pusher or obturator 270 from the coupler 240.

In the illustrated implementation, the pusher or obturator 270 includes an association ramp or bump 274. The association ramp or bump 274 is configured to facilitate the association of the pusher or obturator 270 and the coupler 240 and is configured to help prevent the removal of the pusher or obturator 270 from the coupler 240. For example, in some implementations, the association ramp or bump 274 is configured to contact or engage the coupler 240 to prevent the removal or disassociation of the pusher or obturator 270 from the coupler 240.

The distal end 276 of the pusher or obturator 270 defines a series or slots 273. The slots 273 extend longitudinally. In other words, the slots 273 extend in a direction parallel to a longitudinal axis LA of the pusher or obturator 270. In the illustrated implementation, the pusher or obturator 270 defines six slots. In other implementations, the pusher or obturator 270 defines more than six slots. For example, the distal end portion 276 may define 8, 10, 12 or more slots. In yet other implementations, the pusher or obturator 270 may define less than six slots. For example, the distal end portion 276 may define 1, 2, 3, 4, or 5 slots.

The slots 273 defined by the distal end portion 276 of the pusher or obturator 270 allow the distal end portion 276 of the pusher or obturator 270 to be placed in an expanded configuration and a collapsed configuration. For example, in some implementations, the portions of the pusher or obturator 270 that are disposed adjacent to the slots 273 are configured to flex or bend to allow the outer diameter or other size of the distal end portion to become smaller. In some implementations, when the other surface of the pusher or obturator 270 contacts an inner surface of the elongate member 210, the pusher or obturator 270 may be moved into its contracted or collapsed configuration. In some implementations, portions of the elongate member 210 may have a slightly larger or a slightly smaller inner diameter. These variations may be due to manufacturing processes or due heating and expansion or contraction after the manufacturing process.

As the distal end portion 276 of the pusher or obturator 270 is configured to expand and collapse, the distal end portion 276 of the pusher or obturator 270 can accommodate small changes in the inner diameter of the elongate member 210. Accordingly, as the pusher or obturator 270 is moved within the lumen defined by the elongate member 210 the pusher or obturator 270 may move smoothly even if the inner diameter of the elongate member 210 varies.

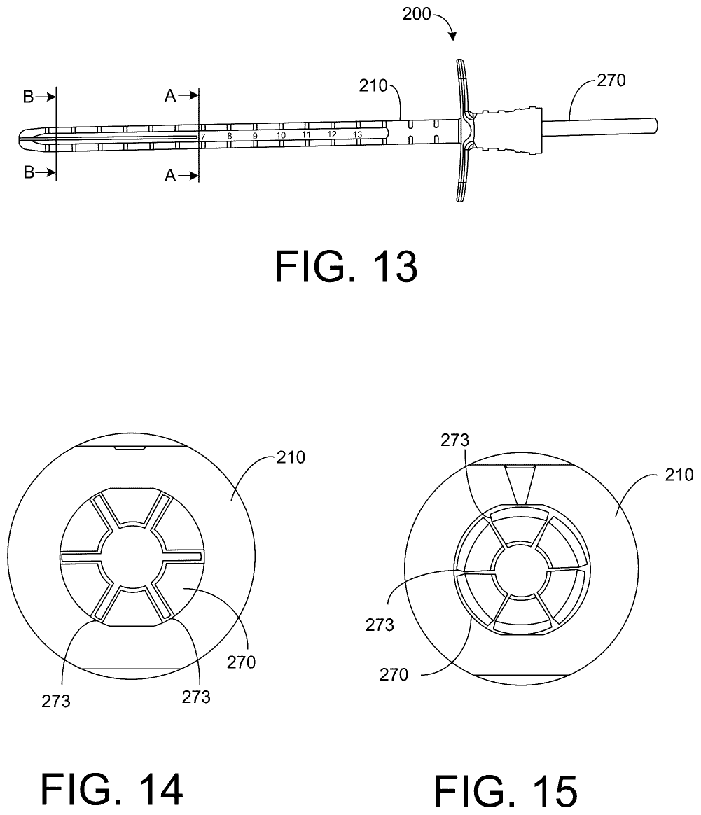

FIG. 13 is a side view of the insertion device 200. FIG. 14 is a cross-sectional view of the insertion device taken along line A-A of FIG. 13. FIG. 15 is a cross-sectional view of the insertion device taken along line B-B of FIG. 13. As best illustrated in FIG. 14, the distal end portion 276 of the pusher or obturator 270 can assume an expanded configuration (larger outer diameter and larger spaces between the portions that are adjacent to the slots) when the distal end portion 276 of the pusher or obturator 270 is located at a location of a larger inner diameter of the elongate member 210. As illustrated in FIG. 15, the distal end portion 276 of the pusher or obturator 270 can assume a collapsed or compressed configuration (smaller outer diameter and smaller spaces between the portions that are adjacent to the slots) when the distal end portion 276 of the pusher or obturator 270 is located at a location of a smaller inner diameter of the elongate member 210.

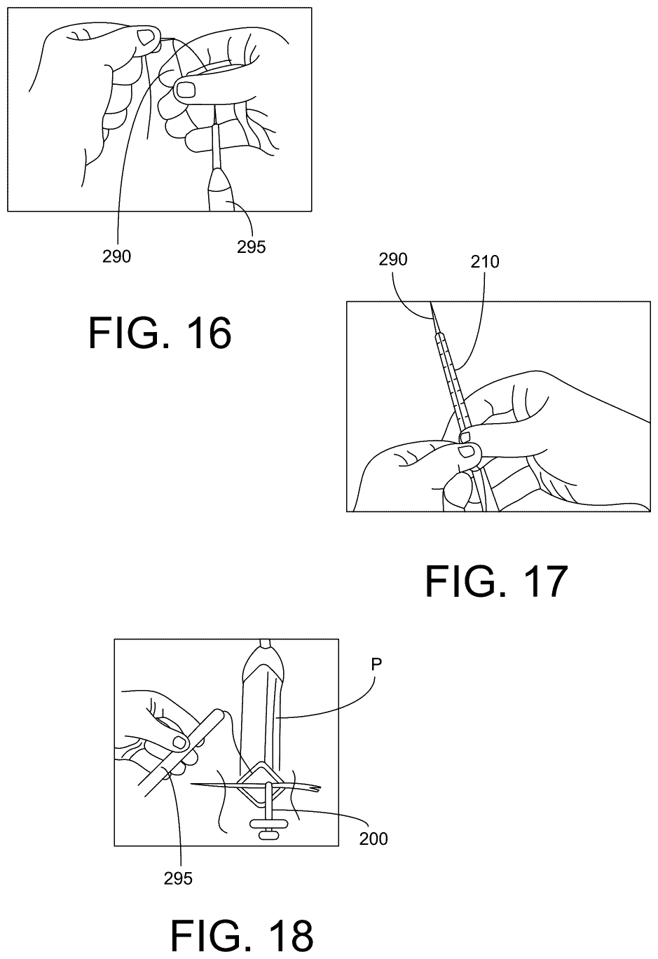

FIGS. 16 to 18 illustrate steps for placing an implant within a body of a patient using a delivery device. As illustrated in FIG. 16, a needle 290 may be associated with or coupled to an implant 195. In some cases, the needle 290 may be associated with the implant 195 via a suture. As illustrated in FIG. 17, the needle 290 may be placed within the lumen defined by the elongate member 210 of the delivery device 200. In some cases, the needle 290 may be inserted into the lumen via a slot defined by the elongate member 210. As illustrated in FIG. 18, the delivery device 200 may be inserted into the body of the patient, such as into a penis P of the patient. Once the delivery device 200 including the needle 290 are placed within the body of the patient at a desired location, the pusher or obturator 270 may then be advanced or moved distally within the lumen defined by the elongate member 210. The pusher or obturator 270 is configured to engage the needle 290 and advance the needle 290 out of the lumen defined by the elongate member 210. The delivery device 200 may then be removed from the body of the patient and the needle 290 can then be pulled or otherwise moved within the body of the patient until the implant 295 is disposed at a desired location.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An insertion device comprising:
an elongate member defining a lumen extending from a first end of the elongate member to a second end of the elongate member; and
a pusher having a first end portion, the first end portion of the pusher being configured to be disposed within the lumen defined by the elongate member and move from a first position within the lumen defined by the elongate member to a second position within the lumen defined by the elongate member, the first end portion of the pusher defining a slot.

2. The insertion device of claim 1, wherein the first end portion of the pusher defines a receiving portion.

3. The insertion device of claim 1, wherein the first end portion of the pusher defines a receiving portion configured to receive a portion of a needle.

4. The insertion device of claim 1, wherein the pusher has a second end portion opposite the first end portion, the second end portion includes a handle portion.

5. The insertion device of claim 1, wherein the pusher has a second end portion opposite the first end portion, the second end portion includes a handle portion, the handle portion being configured to extend from the lumen defined by the elongate member.

6. The insertion device of claim 1, wherein the pusher has a second end portion opposite the first end portion, the second end portion includes a handle portion, the handle portion being configured to extend from the lumen defined by the elongate member while the first end portion of the pusher is disposed within the lumen defined by the elongate member.

7. The insertion device of claim 1, wherein the slot defined by the first end portion of the pusher extends in a direction parallel to a longitudinal axis of the pusher.

8. The insertion device of claim 1, wherein the slot defined by the first end portion of the pusher is a first slot, the first end portion of the pusher defining a second slot.

9. The insertion device of claim 1, wherein the slot defined by the first end portion of the pusher is a first slot, the first end portion of the pusher defining a second slot, the first slot extends in a direction parallel to a longitudinal axis of the pusher, and the second slot extends in a direction parallel to the longitudinal axis of the pusher.

10. The insertion device of claim 1, wherein the first end portion of the pusher defines a plurality of slots.

11. The insertion device of claim 1, wherein the slot defined by the first end portion of the pusher is a first slot, the first end portion of the pusher defining a second slot, a third slot, and a fourth slot.

12. The insertion device of claim 1, wherein the first end portion of the pusher is configured to be placed in an expanded configuration and a collapsed configuration.

13. The insertion device of claim 1, wherein the first end portion of the pusher is configured to be placed in an expanded configuration and a collapsed configuration, the first end portion of the pusher being configured to be placed in the collapsed configuration in response to the first end portion of the pusher contacting an inner diameter of the elongate member.

14. The insertion device of claim 1, wherein the first end portion of the pusher includes a first section disposed on a first side of the slot and a second section disposed on a second side of the slot, the first end portion of the pusher is configured to be placed in an expanded configuration and a collapsed configuration, the first section of the pusher being disposed closer to the second section of the pusher when the first end portion of the pusher is disposed in the collapsed configuration than when the first end portion of the pusher is disposed in the expanded configuration.

15. The insertion device of claim 1, wherein the elongate member defines a slot.

16. A kit, comprising:
a bodily implant;
a delivery needle configured to be coupled to the bodily implant; and
a delivery device having an elongate member defining a lumen extending from a first end of the elongate member to a second end of the elongate member, and a pusher having a first end portion, the first end portion of the pusher being configured to be disposed within the lumen defined by the elongate member and move from a first position within the lumen defined by the elongate member to a second position within the lumen defined by the elongate member, the first end portion of the pusher defining a slot.

17. The kit of claim 16, wherein the elongate member defines a slot configured to receive at least a portion of the delivery needle.

18. The kit of claim 16, wherein the slot defined by the first end portion of the pusher extends in a direction parallel to a longitudinal axis of the pusher.

19. The kit of claim 16, wherein the slot defined by the first end portion of the pusher is a first slot, the first end portion of the pusher defining a second slot.

20. The kit of claim 16, wherein the slot defined by the first end portion of the pusher is a first slot, the first end portion of the pusher defining a second slot, the first slot extends in a direction parallel to a longitudinal axis of the pusher, and the second slot extends in a direction parallel to the longitudinal axis of the pusher.
